(19) United States
(12) Patent Application Publication
(10) Pub. No.: US 2008/0293805 A1
(43) Pub. Date: Nov. 27, 2008

(54) DRUG DELIVERY SYSTEM AND THE PREPARING METHOD THEREOF

(76) Inventor: Shu-Yi Lin, Hsinchu City (TW)

Correspondence Address: SINORICA, LLC, 528 FALLSGROVE DRIVE, ROCKVILLE, MD, 20850, US

(21) Appl. No.: 12/113,931

(22) Filed: May 1, 2008

(30) Foreign Application Priority Data
May 25, 2007 (TW) 096118654

(51) Int. Cl. A61K 9/127; A61K 47/08

(52) U.S. Cl. 514/450; 514/772

(57) ABSTRACT

A drug delivery system comprises a liposome and at least one charged nanoparticle. The liposome has at least an internal lipid layer and an external lipid layer, wherein the internal lipid layer forms an interior space for accommodating a drug. The charged nanoparticle is reversibly associated with an exterior surface of the external lipid layer of the liposome.

### BACKGROUND OF THE INVENTION

1. Field of the Invention

The present invention relates to a drug delivery system, and especially relates to a stable liposome drug delivery system.

2. Descriptions of the Related Art

Liposomes are vesicles composed of phosoholipids, wherein most liposomes are natural materials so they are nontoxic and biodegradable.

As shown in FIG. 1, the liposome 100 is composed of phosoholipid molecules 105, wherein the phosphoric acid head 110 of the phosoholipid molecule 105 is hydrophilic and the fatty acid tail 112 thereof is hydrophobic. When the phosoholipid molecules are introduced into an aqueous environment, a liposome 100 is formed, which has a bilayer structure 102 with outward phosphoric acid heads 110 and inward fatty acid tails 112, and thereby a roughly hollow sphere structure is provided. As a result, a hydrophilic substance will be entrapped in the hollow portion of the sphere and a hydrophobic substance will be incorporated in the bilayer membrane, and, thereby, liposomes can be used in a drug delivery system as carriers either for hydrophilic or hydrophobic compounds.

The objective of a drug delivery system is to make the drugs comprise the properties of controlled release and targeting. After the drug delivery system gets into an organism, the drugs will not be released before arriving its target region or tissue. When the drugs arrive the target region or tissue, an effective amount thereof is released in a predetermined velocity and sustained for a desired period of time. Such design can prevent the drugs from damaging healthy tissues or organs and then reduce the needed dosage and frequency of taking medicines.

There are some commercial liposome drugs. Vincristine is a drug for treatment of leukemia. Combining vincristine and liposome enhances its efficacy as well as lessens other side effects, such as nausea, dizziness and loss of hair. Besides, the combination also prevents the drugs from accumulating in high concentrations in susceptible organs such as the kidneys and liver.

Phospholipid bilayer does not come into being liposomal vesicle spontaneously. Therefore, either a physical or a chemical method is necessay to produce the needed liposomes from hydrated lipids. Introducing high energies (such as high-heat, high-pressure, and ultrasound) are common methods to disperse the low critical micelle concentration phospholipids as a metastable liposome state, and the needed liposomes are produced thereafter.

However, unstable liposomes tend to disrupt easily. “Emulsion stability” generally refers to the ability of an emulsion (e.g. liposome) to resist changes in structure with time. The sedimentation and creaming are both structural changes resulting from gravity. Sedimentation is resulted from the density of liposomes is greater than that of its surrounding solution. If the density of liposomes is smaller than that of its surrounding solution, creaming is occurred. “Flocculation” and “coagulation” are both the results of liposome aggregation, wherein the flocculation is a reversible liposome polymerization and the coagulation is an irreversible one.

Referring to FIGS. 2A to 2D, the liposome 210 carries drugs A and the liposome 220 carries drugs B. As these two liposomes approach and then contact to each other, the lipid membranes in the contact region will collapse and fuse. A new liposome 230 will be formed eventually, and the drugs carried by the liposome 230 are the mixture of drugs A and drugs B. If this scenario occurs in the organism, it may lead to drug composition changes or premature drug release before arriving their targets, and both will cause adverse effects to organisms.

Therefore, the commercial liposomes can be used in the organism. Their designs are nonetheless insufficient for a drug delivery system. It has to be more accurate for a drug delivery system, that is, liposomes in this case, to control both where and when to release the drugs. The more stable liposomes, the better pharmacokinetics they will have.

Therefore, a drug delivery system and the preparation thereof disclosed in the present invention will solve the aforementioned defects.

### SUMMARY OF THE INVENTION

The primary object of this invention is to provide a drug delivery system, wherein the liposome used for accommodating the drugs has a stable structure and will not integrate with each other.

Another object of this invention is to provide a drug delivery system, wherein an external layer of the liposome used for accommodating the drug comprises charged nanoparticles that are reversibly associated; furthermore, the nanoparticles are nontoxic and can be excreted by the organism via the metabolism.

Another object of this invention is to provide a drug delivery system, wherein an external layer of liposome used for accommodating the drug comprises charged nanoparticles that are reversibly associated; furthermore, the association of the nanoparticles and the liposomes can be controlled by changing the pH value of the environment.

Still another object of the present invention is to provide a drug delivery system wherein liposomes carrying a drug can be associated to charged nanoparticles through simple procedures.

Yet another object of this invention is to provide a drug delivery system which comprises a liposome and at least one charged nanoparticle, wherein the liposome comprises at least one internal lipid layer and one external lipid layer. The internal lipid layer forms an interior space for accommodating the drug, and the charged nanoparticle is reversibly associated with the exterior surface of the external lipid layer.

The invention further provides a method for manufacturing a drug delivery system, which comprises the following steps: providing a plurality of charged nanoparticles; providing a solution comprising at least one category of liposomes, and mixing the charged nanoparticles with the solution comprising the liposome.

### BRIEF DESCRIPTION OF THE DRAWINGS

FIG. 1 is a sectional drawing of a liposome structure;

FIGS. 2A to 2D illustrate the membrane fusion process of liposomes;

FIG. 3 is a chart of the drug delivery system of the preferred embodiment according to the present invention;

FIG. 4 is another chart of the drug delivery system of the preferred embodiment according to the present invention.

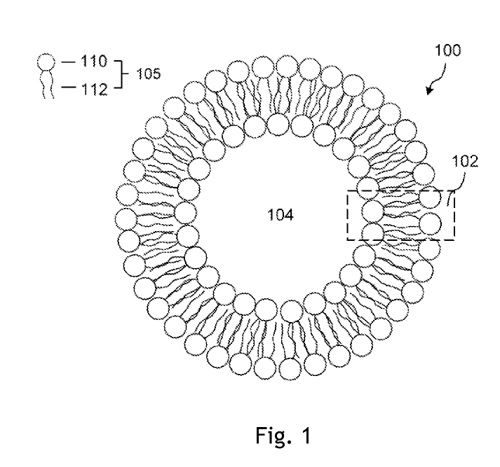

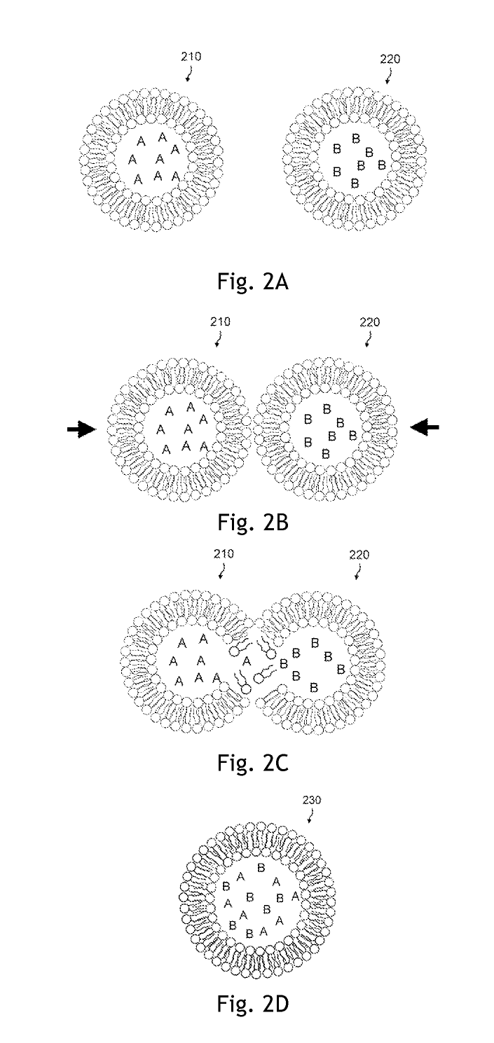

### DESCRIPTION OF THE PREFERRED EMBODIMENT

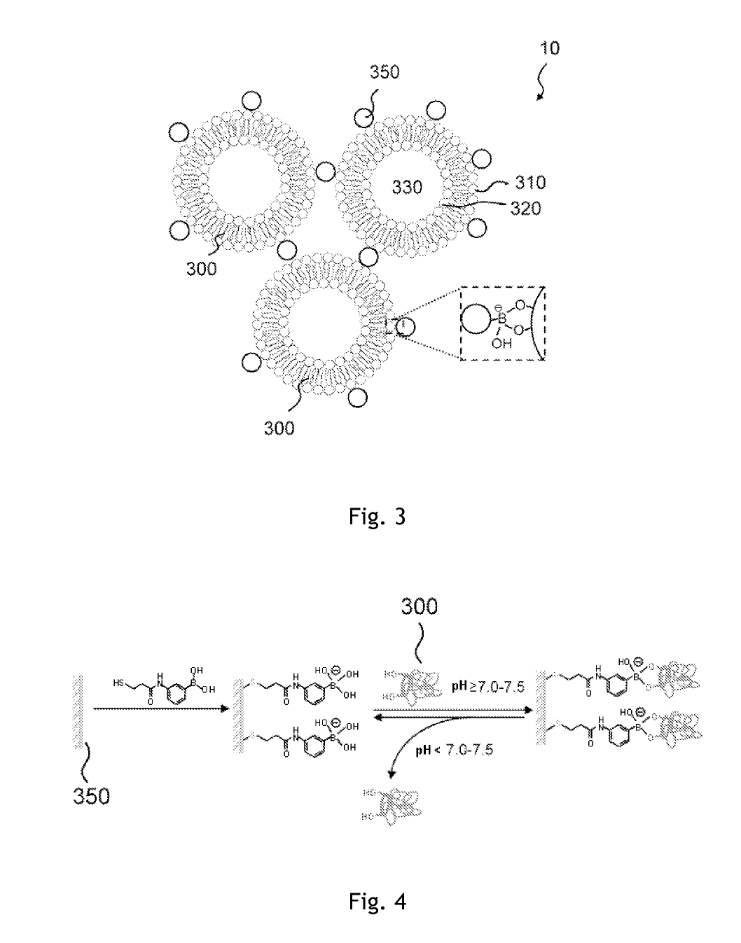

A drug delivery system 10 of one preferred embodiment according to the present invention is provided, wherein the drugs could be pharmaceutical active ingredients, peptides, proteins, nucleic acids, polynucleotides, plasmids or synthetic chemical drugs. As shown in FIG. 3, the system 10 comprises a liposome 300. The liposome 300 comprises at least one internal lipid layer 320 and an external lipid layer 310. Besides, the internal lipid layer 320 forms an interior space 330 for accommodating drugs while at least one charged nanoparticle 350 is associated with the exterior surface of the external lipid layer 310. The nanoparticle 350 could be macromolecules, metals, carbons, or the combination thereof. More preferably, the nanoparticle 350 could be gold, silver, or copper. The nanoparticle 350 can be reversibly associated with the external lipid layer 310. The nanoparticle 350 is usually negatively charged, so that the liposomes can be repulsed from each other, which can avoid the disruption resulted from the membrane fusion.

In another preferred embodiment according to the present invention, as shown in FIG. 4, the liposome 300 is composed of phospholipids conjugated with carbohydrates, and the exterior surface of the external lipid layer of the liposome comprises diols. If the surface of the nanoparticles 350 conjugate with at least one single layer of borates, the nanoparticles 350 can attach the external lipid layer via the good bonding effect of borates and diols. The bonding effect changes with the pH value of the environment. When the pH value of the environment is equal to or greater than a number and the number is between about 7.0 and about 7.5, the bonding of the nanoparticles 350 and the liposome 300 is most stable. If the pH value of the environment is smaller than a number and the number is between about 7.0 and about 7.5, the nanoparticles 350 will detach from the liposome 300.

If the phospholipids of the liposome do no conjugated with carbohydrates, the nanoparticles 350 preferably conjugate with compounds having carboxyl groups instead. Since the carboxyl groups are partial negatively charged and the heads of the phospholipids are partial positively charged, the nanoparticles and the liposomes can thereby associate with each other via the attraction forces exerted by the positive and negative electric charges of the liposomes and nanoparticles, respectively. Professor Retello of University of Massachusetts-Amherst has demonstrated a toxicity test of gold-nanoparticles with different electric charges, which suggested that the gold-nanoparticles conjugated with the compounds having carboxyl groups are nontoxic. The gold-nanoparticles are found that they will not accumulate in the organism and the organism can excrete them via the metabolism. Therefore, the gold-nanoparticles conjugated with the compounds having carboxyl groups are much safe to be used in a drug delivery system.

Since the nanoparticles 350 are very tiny, they can not only be applied to the liposomes with a diameter ranging from 200 nm to 500 nm, but can be also applied to the liposomes with a diameter ranging from 2 nm to 50 nm. In other words, the nanoparticles 350 are suitable for the delivery of all liposome drugs.

A method for manufacturing a drug delivery system is also disclosed in the present invention, which comprises the following steps. First, provide a plurality of charged nanoparticles and a solution containing at least one category of liposomes, followed by mixing the charged nanoparticles and the liposome solution.

The step of providing the nanoparticles includes providing a plurality of nanoparticle matrixes and solutions of capping agents, and mixing the nanoparticle matrixes and the solutions of capping agents.

The capping agents can conjugate at least one single layer of compounds on the surface of the nanoparticles. The formula of the compounds contains borates, thiols, or both of them. Thiols can bind to the surface of the nanoparticles easily and stably, and borates can form stable bonds with diols of the phospholipids. Together, the nanoparticles conjugated with a single layer of compounds with borates and/or thiols are able to bind the liposome easily.

The capping agents can further comprise compounds with carboxyl groups. The carboxyl groups are partial negatively charged and the heads of the phospholipids are partial positively charged. If the heads of the phospholipids of the liposomes conjugate with compounds having carboxyl groups, the nanoparticles can reversibly associate with the liposomes via the attraction force exerted by the negative and positive electric charges of the nanoparticles and the liposomes, respectively.

The present invention provides a more stable liposome that will not fuse with each other easily. Meanwhile, changing the pH value of the environment can control the association and dissociation of the nanoparticles and the liposomes. By choosing a suitable category of nanoparticles, the non-medical nanoparticles can be voided by the metabolism of the organism and will not burden the organism.

While the invention has been described by way of examples and in terms of the preferred embodiments, it is to be understood that the invention is not limited to the disclosed embodiments. To the contrary, it is intended to cover various modifications and similar arrangements as would be apparent to those skilled in the art. Therefore, the scope of the appended claims should be accorded the broadest interpretation so as to encompass all such modifications and similar arrangements.

What is claimed is:

1. A drug delivery system, comprising:
a liposome, which comprises at least one internal lipid layer and one external lipid layer, in which the internal lipid layer forms an interior space for accommodating a drug; and
at least one charged nanoparticle, for being reversibly associated with an exterior surface of the external lipid layer.

2. The drug delivery system according to claim 1, wherein the nanoparticle is generally chosen from the group consisting of macromolecules, metals, carbons and combinations thereof.

3. The drug delivery system according to claim 1, wherein the nanoparticle is generally chosen from the group consisting of gold, silver and copper.

4. The drug delivery system according to claim 1, wherein the nanoparticle is negatively charged.

5. The drug delivery system according to claim 1, wherein the external lipid layer comprises diols on the exterior surface.

6. The drug delivery system according to claim 1, wherein the nanoparticle was conjugated with at least one single layer of compounds with borates.

7. The drug delivery system according to claim 6, wherein the nanoparticle is most stably associated with the liposome while the pH value of the environment is equal to or greater than a number, which is between about 7.0 and about 7.5.

8. The drug delivery system according to claim 6, wherein the nanoparticle is dissociated from the liposome while the pH value of the environment is smaller than a number, which is between about 7.0 and about 7.5.

9. The drug delivery system according to claim 1, wherein the nanoparticle was conjugated with at least one single layer of compounds with carboxyl groups.

10. The drug delivery system according to claim 1, wherein the liposome has a diameter ranging from 200 nm to 500 nm.

11. The drug delivery system according to claim 1, wherein the liposome has a diameter ranging from 2 nm to 50 nm.

12. A method for manufacturing a drug delivery system, comprising:
providing a plurality of charged nanoparticles;
providing a solution which comprises at least one category of liposomes; and
mixing the charged nanoparticles and the solution comprising the liposome.

13. The method according to claim 12, wherein the nanoparticle is generally chosen from the group consisting of macromolecules, metals, carbons and combinations thereof.

14. The method according to claim 12, wherein the nanoparticle is generally chosen from the group consisting of gold, silver and copper.

15. The method according to claim 12, wherein the step of providing nanoparticles comprises:
providing a plurality of nanoparticle matrixes;
providing a solution of capping agent; and
mixing the nanoparticle matrixes and the solution of capping agent.

16. The method according to claim 15, wherein the solution of capping agent comprises boric acids and thiols.

17. The method according to claim 15, wherein the solution of capping agent comprises compounds having borate ligands.

18. The method according to claim 15, wherein the solution of capping agent comprises compounds having carboxyl groups.

19. The method according to claim 12, wherein a hydrophilic part of the liposome comprises diols.
